(19) United States
(12) Patent Application Publication
(10) Pub. No.: US 2025/0248613 A1
(43) Pub. Date: Aug. 7, 2025

(54) LOCALIZATION METHOD FOR CEREBRAL INFARCTION AREA, SYSTEM, MEDIUM, AND PRODUCT

(71) Applicant: Anying Technology (Beijing) Co., Ltd., Beijing (CN)

(72) Inventors: Gang MENG, Beijing (CN); Ligeng LI, Beijing (CN); Mingyang ZHAO, Beijing (CN); Xiangyu GONG, Beijing (CN); Lihua ZHONG, Beijing (CN)

(73) Assignee: Anying Technology (Beijing) Co., Ltd., Beijing (CN)

(21) Appl. No.: 18/757,956

(22) Filed: Jun. 28, 2024

(30) Foreign Application Priority Data
Feb. 4, 2024 (CN) 202410155364.0

(51) Int. Cl. A61B 5/026; A61B 5/00; A61B 5/055; G01R 33/56; G01R 33/563

(52) U.S. Cl. CPC A61B 5/0263; A61B 5/0042; A61B 5/055; G01R 33/5608; G01R 33/56366

(57) ABSTRACT

Please cancel the Abstract and replace the Abstract with the following text: The invention relates to a localization method for a cerebral infarction area, a system, a non-transitory computer readable storage, and a product, relating to medical image processing. The method includes taking the highest cerebral blood flow of each preset segmentation unit in a brain area as corrected cerebral blood flow of each preset segmentation unit based on a cerebral blood perfusion image with multiple post-labeling delays; and performing threshold gradient segmentation according to the corrected cerebral blood flow of each preset segmentation unit, so as to obtain a gradient segmentation result. The accuracy of localizing the cerebral infarction area is improved.

### CROSS-REFERENCE TO RELATED APPLICATION

This patent application claims the benefit and priority of Chinese Patent Application No. 202410155364.0 filed with the China National Intellectual Property Administration on Feb. 4, 2024, the disclosure of which is incorporated by reference herein in its entirety as part of the present application.

### BACKGROUND OF THE INVENTION

### Field of the Invention

At least one embodiment of the invention relates to the technical field of medical imaging processing, and in particular to a localization method for a cerebral infarction area, a system, a medium, and a product.

### Description of the Related Art

In existing methods, the infarct lesion area is determined by local cerebral blood flow or comparing relative differences between the left and right sides, but the accuracy is poor when there are lesions on both sides of the brain area of patient or a hyper-perfusion signal occurs on contralateral side due to compensation, or the characteristics of abnormal structures in the structural diagram are directly observed by naked eyes, but the observation results cannot be quantified and is limited to the professional level of the observer. Therefore, the accuracy of localizing the cerebral infarction area needs to be improved.

### BRIEF SUMMARY OF THE INVENTION

An objective of at least one embodiment of the invention is to provide a localization method for a cerebral infarction area, a system, a medium, and a product, so as to improve the accuracy of localizing the cerebral infarction area.

To achieve the objective above, at least one embodiment employs the following technical solution:
A localization method for a cerebral infarction area includes:
taking the highest cerebral blood flow of each preset segmentation unit in a brain region as corrected cerebral blood flow of each preset segmentation unit based on a cerebral blood perfusion image with multiple post-labeling delays;
performing threshold gradient segmentation according to the corrected cerebral blood flow of each preset segmentation unit, so as to obtain a gradient segmentation result; and
determining the cerebral infarction area according to the gradient segmentation result.

Alternatively, in one or more embodiments, taking the highest cerebral blood flow of each preset segmentation unit in the brain region as corrected cerebral blood flow of each preset segmentation unit based on the cerebral blood perfusion image with multiple post-labeling delays specifically includes:
importing the cerebral blood perfusion image to be localized into a structural space to obtain a cerebral blood perfusion model, where the cerebral blood perfusion image to be localized is a cerebral blood perfusion image comprising multiple post-labeling delays;
registering a brain atlas to the cerebral blood perfusion model to obtain a blood perfusion model of a brain partition; and
taking the highest cerebral blood flow in the multiple post-labeling delays corresponding to each preset segmentation unit in each partition in the blood perfusion model of the brain partition as corrected cerebral blood flow of each preset segmentation unit.

Alternatively, in one or more embodiments, prior to importing the cerebral blood perfusion image to be localized into the structural space to obtain the cerebral blood perfusion model, the method further includes:
acquiring a cerebral blood perfusion image of an individual brain by using a multi-delay pseudo-continuous arterial spin labeling method.

Alternatively, in one or more embodiments, the structural space is a T2 Flair space.

Alternatively, in one or more embodiments, the brain atlas comprises an AAL3 atlas of MNI152 space, left and right brain atlases, and ASPECTS atlas.

Alternatively, in one or more embodiments, in the gradient segmentation result, for each brain partition:
an area with the cerebral blood flow greater than or equal to 0 and less than 10 is determined as a core infarct area;
an area with the cerebral blood flow greater than or equal to 10 and less than 20 is determined as an ischemic penumbra;
an area with the cerebral blood flow greater than or equal to 20 and less than 40 is determined as a hypoperfusion area;
an area with the cerebral blood flow greater than or equal to 40 and less than 100 is determined as a normal perfusion area;
an area with the cerebral blood flow greater than or equal to 100 is determined as a hyper-perfusion area.

Alternatively, in one or more embodiments, the core infarct area, the ischemic penumbra, the hypoperfusion area and the normal perfusion area are distinguished and displayed by colors.

A computer system is further provided by at least one embodiment of the invention, including a memory, a processor, and a computer program stored on the memory and capable of being operated on the processor. The processor, is configured to execute the computer program, is configured to achieve steps of the localization method for the cerebral infarction area, according to one or more embodiments of the invention.

A non-transitory computer-readable storage medium is further provided by at least one embodiment of the invention. A computer program is stored on the non-transitory computer readable storage medium, and the computer program, when executed by a processor, can achieve steps of the localization method for the cerebral infarction area, according to one or more embodiments of the invention.

A computer program product is further provided by at least one embodiment of the invention, including a computer program. The computer program, when executed by a processor, can achieve steps of the localization method for the cerebral infarction area.

According to one or more embodiments of the invention, the one or more embodiments have the following technical effects:

The highest cerebral blood flow of each preset segmentation unit in a brain area is used as the corrected cerebral blood flow of each preset segmentation unit based on a cerebral blood perfusion image with multiple post-labeling delays to achieve partitioned correction of the cerebral blood, thus acquiring accurate cerebral blood and achieving accurate localization of the cerebral infraction area.

### BRIEF DESCRIPTION OF THE DRAWINGS

To describe the technical solutions of the one or more embodiments of the invention or in the prior art more clear, the following briefly introduces the accompanying drawings required for describing the one or more embodiments. Apparently, the accompanying drawings in the following description show merely some embodiments of the invention, and those of ordinary skill in the art may still derive other drawings from these accompanying drawings without creative efforts.

FIG. 1 is a flow diagram of a localization method for a cerebral infarction area according to one or more embodiments of the invention;

FIG. 2 is a diagram of an internal structure of a computer system, according to one or more embodiments of the invention.

### DETAILED DESCRIPTION OF THE INVENTION

The following clearly and completely describes the technical solutions in the one or more embodiments of the invention with reference to the accompanying drawings in the one or more embodiments of the invention. Apparently, the described one or more embodiments are merely a part rather than all of the embodiments of the invention. All other embodiments obtained by a person of ordinary skill in the art based on the one or more embodiments of the invention without creative efforts shall fall within the protection scope of the invention.

An objective of at least one embodiment of the invention is to provide localization method for a cerebral infarction area, a system, a medium, and a product, so as to improve the accuracy of localizing the cerebral infarction area.

In order to make the above objectives, features and advantages of one or more embodiments of the invention more clearly, the one or more embodiments of the invention is further described in detail below with reference to the accompanying drawings and specific embodiments.

### Example 1

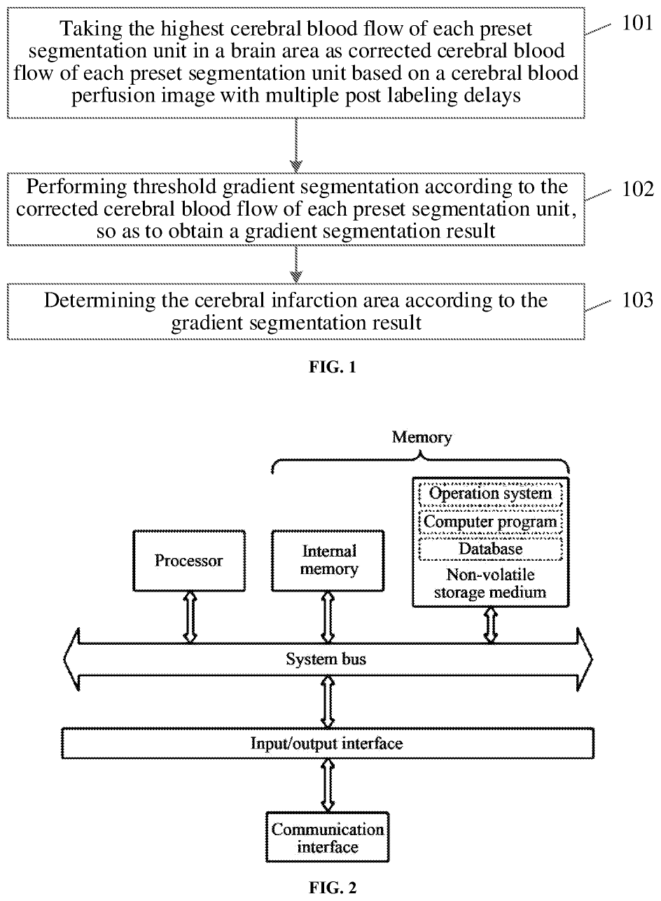

As shown in FIG. 1, a localization method for a cerebral infarction area by way of one or more embodiments includes the following steps:
Step 101: Based on a cerebral blood perfusion image with multiple post-labeling delays, the highest cerebral blood flow of each preset segmentation unit in a brain area is used as corrected cerebral blood flow of each preset segmentation unit.
Step 102: The brain area is subjected to threshold gradient segmentation according to the corrected cerebral blood flow of each preset segmentation unit, so as to obtain a gradient segmentation result.
Step 103: A cerebral infarction area is determined according to the gradient segmentation result.

Cerebral blood flow (CBF) is the number of milliliters of blood flow per minute per 100 g of brain tissue.

Step 101 specifically includes:

A cerebral blood perfusion image to be localized and a corresponding arterial transit time image are imported into a structural space to obtain a cerebral blood perfusion model, where the cerebral blood perfusion image to be localized is a cerebral blood perfusion image including multiple post-labeling delays.

A brain atlas is registered to the cerebral blood perfusion model to obtain a blood perfusion model of a brain partition.

The brain atlas is used to partition the brain tissue, where each brain partition includes the parietal lobe, frontal lobe, occipital lobe, and temporal lobe.

The highest cerebral blood flow in multiple post-labeling delays (PLD) corresponding to each preset segmentation unit in each partition in the blood perfusion model of the brain partition is used as corrected cerebral blood flow of each preset segmentation unit.

Arterial transit time (ATT) corresponding to the highest cerebral blood flow in the multiple post-labeling delays corresponding to each preset segmentation unit is used as the optimal arterial transit time of each preset segmentation unit. The preset segmentation unit is at least one pixel, and the size of the preset segmentation unit can be set according to actual needs.

Prior to importing the cerebral blood perfusion image to be localized into the structural space to obtain the cerebral blood perfusion model, the method further includes:

A cerebral blood perfusion image and an arterial transit time image of an individual brain are acquired by using a multi-delay pseudo-continuous arterial spin labeling (pCASL) method.

The structural space is a T2 Flair space.

The brain atlas includes an AAL3 atlas of MNI152 space, cerebral lobe atlas, left and right brain atlases, and ASPECTS atlas.

The left and right brain atlases divide the T2 Flair space into two perfusion areas, i.e., a left perfusion area, and a right perfusion area, respectively.

The multiple post-labeling delays are five post-labeling delays, and the occurrence time of the five post-labeling delays are 0.5 s, 1 s, 1.5 s, 2 s and 2.5 s in turn, or the multiple post-labeling delays include any two or three of 1.0 s, 1.5 s, 2 s and 2.5 s. That is, the number of the post-labeling delays may be five, or may also be two, or three.

In at least one embodiment, a new generation of 4DASL (Multi-delay pCASL) scanning sequence and Cereflow efficient computing platform system are adopted to solve the technical limitations of ASL (arterial spin labeling), and the corrected CBF calculation of 300 target areas (partitions) of the whole brain in 30-90 seconds is provided.

In the gradient segmentation result, for each brain partition:
an area with the cerebral blood flow greater than or equal to 0 and less than 10 is determined as a core infarct area;
an area with the cerebral blood flow greater than or equal to 10 and less than 20 is determined as ischemic penumbra;
an area with the cerebral blood flow greater than or equal to 20 and less than 40 is determined as a hypoperfusion area;
an area with the cerebral blood flow greater than or equal to 40 and less than 100 is determined as a normal perfusion area; and
an area with the cerebral blood flow greater than or equal to 100 is determined as a hyper-perfusion area.

The cerebral blood flow area takes the preset segmentation unit as the minimum unit.

The core infarct area, the ischemic penumbra, the hypoperfusion area and the normal perfusion area are distinguished and displayed by colors.

The localization method for the cerebral infarction area of one or more embodiments of the invention further includes: the arterial transit time corresponding to the highest cerebral blood flow among the multiple post-labeling delays corresponding to each area is taken as the optimal arterial transit time of each area.

If the optimal arterial transit time of area i exceeds 1.3 times the set arterial transit time of area i, there is collateral circulation in area i. By this determination, the collateral circulation is established in this area, and there is compensatory circulation. The set arterial transit time of the area i is the standard arterial transit time of area i.

In at least one embodiment, the localization method for the cerebral infarction area further includes calculating a volume ratio of an IsP area and an IsC area, and a volume composition ratio of IsP area/(IsP area+IsC area). The IsP area is the ischemic penumbra, and the IsC area is the core infarct area.

Based on the corrected CBF, the results of the infarct area of CBF in each partition and even each pixel can be obtained by the present disclosure, and the accurate localization of the cerebral infarction area can be achieved.

### Example 2

A diagram of an internal structure of a computer system provided by one or more embodiments of the invention may be shown in FIG. 2. The computer system includes a processor, memory, an input/output interface (short for I/O), and a communication interface. Herein, the processor, the memory and the input/output are connected through a system bus, and the communication interface is connected to the system bus through the input/output interface. The processor of the computer system is used to provide computing and control capabilities. The memory of the computer system includes a non-volatile storage medium, and an internal memory. An operation system, a computer program and a database are stored in the non-volatile storage medium. The internal memory provides an environment for the running of the operation system and the computer program in the non-volatile storage medium. The database of the computer system is used for storing pending transactions. The input/output interface of the computer system is used for exchanging information between the processor and an external device. The communication interface of the computer system is used for communicating with an external terminal through a network. The computer program, when executed by the processor, can achieve steps of the localization method for a cerebral infarction area provided according to one or more embodiments of the invention.

### Example 3

A non-transitory computer-readable storage medium is further provided by one or more embodiments of the invention, a computer program is stored on the non-transitory computer readable storage medium, and the computer program, when executed by a processor, can achieve steps of the localization method for a cerebral infarction area by way of at least one embodiment of the invention.

### Example 4

A computer program product is further provided by at least one embodiment of the invention, including a computer program. The computer program, when executed by a processor, can achieve steps of the localization method for a cerebral infarction area according to one or more embodiments of the invention.

The technical features of the one or more embodiments can be combined at will. In order to make the description concise, not all possible combinations of the technical features in the above embodiments are described. However, it should be considered that these combinations of technical features fall within the scope recorded in this specification provided that these combinations of technical features do not have any conflict.

Specific examples are used here for illustration of the principles and implementation methods of one or more embodiments of the invention. The description of the above embodiments is merely used to help illustrate the method and its core principles of the invention. In addition, a person of ordinary skill in the art can make various modifications in terms of specific embodiments and scope of application in accordance with the teachings of the invention. In conclusion, the content of this specification shall not be construed as a limitation to the one or more embodiments of the invention.

1. A localization method for a cerebral infarction area, comprising:
taking a highest cerebral blood flow of each preset segmentation unit in a brain area as corrected cerebral blood flow of said each preset segmentation unit based on a cerebral blood perfusion image with multiple post-labeling delays;
performing threshold gradient segmentation according to the corrected cerebral blood flow of each preset segmentation unit, so as to obtain a gradient segmentation result; and
determining the cerebral infarction area according to the gradient segmentation result.

2. The localization method for the cerebral infarction area according to claim 1, wherein taking the highest cerebral blood flow of each preset segmentation unit in the brain area as corrected cerebral blood flow of each preset segmentation unit based on the cerebral blood perfusion image with multiple post-labeling delays comprises:
importing the cerebral blood perfusion image to be localized into a structural space to obtain a cerebral blood perfusion model, wherein the cerebral blood perfusion image to be localized is the cerebral blood perfusion image comprising said multiple post-labeling delays;
registering a brain atlas to the cerebral blood perfusion model to obtain a blood perfusion model of a brain partition; and
taking the highest cerebral blood flow in the multiple post-labeling delays corresponding to said each preset segmentation unit in each partition in the blood perfusion model of the brain partition as said corrected cerebral blood flow of said each preset segmentation unit.

3. The localization method for the cerebral infarction area according to claim 2, wherein prior to said importing the cerebral blood perfusion image to be localized into the structural space to obtain the cerebral blood perfusion model, the localization method further comprises:
acquiring a cerebral blood perfusion image of an individual brain by using a multi-delay pseudo-continuous arterial spin labeling method.

4. The localization method for the cerebral infarction area according to claim 2, wherein the structural space is a T2 Flair space.

5. The localization method for the cerebral infarction area according to claim 2, wherein the brain atlas comprises an AAL3 atlas of MNI152 space, left and right brain atlases, and ASPECTS atlas.

6. The localization method for the cerebral infarction area according to claim 2, wherein in the gradient segmentation result, for each brain partition, comprises:
an area with a cerebral blood flow greater than or equal to 0 and less than 10 is determined as a core infarct area;
an area with the cerebral blood flow greater than or equal to 10 and less than 20 is determined as an ischemic penumbra;
an area with the cerebral blood flow greater than or equal to 20 and less than 40 is determined as a hypoperfusion area;
an area with the cerebral blood flow greater than or equal to 40 and less than 100 is determined as a normal perfusion area; and
an area with the cerebral blood flow greater than or equal to 100 is determined as a hyper-perfusion area.

7. The localization method for the cerebral infarction area according to claim 6, wherein the core infarct area, the ischemic penumbra, the hypoperfusion area and the normal perfusion area are distinguished and displayed by colors.

8. A computer system, comprising:
a memory,
a processor, and
a computer program stored on the memory and capable of being operated on the processor,
wherein the processor, is configured to execute the computer program to achieve a localization method for a cerebral infarction area, said localization method comprising
taking a highest cerebral blood flow of each preset segmentation unit in a brain area as corrected cerebral blood flow of said each preset segmentation unit based on a cerebral blood perfusion image with multiple post-labeling delays;
performing threshold gradient segmentation according to the corrected cerebral blood flow of each preset segmentation unit, so as to obtain a gradient segmentation result; and
determining the cerebral infarction area according to the gradient segmentation result.

9. A non-transitory computer readable storage medium, wherein a computer program is stored on the non-transitory computer readable storage medium, and the computer program, when executed by a processor, is able to achieve a localization method for a cerebral infarction area, said localization method comprising:
taking a highest cerebral blood flow of each preset segmentation unit in a brain area as corrected cerebral blood flow of said each preset segmentation unit based on a cerebral blood perfusion image with multiple post-labeling delays;
performing threshold gradient segmentation according to the corrected cerebral blood flow of each preset segmentation unit, so as to obtain a gradient segmentation result; and
determining the cerebral infarction area according to the gradient segmentation result.

10. (canceled)
